(19) United States
(12) Patent Application Publication
(10) Pub. No.: US 2007/0265238 A1
(43) Pub. Date: Nov. 15, 2007

(54) METHOD FOR ACCELERATING REENTRAINMENT OF A CIRCADIAN RHYTHM

(76) Inventor: Tammy Jechura, Albion, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/674,785

(22) Filed: Feb. 14, 2007

Related U.S. Application Data
(60) Provisional application No. 60/773,801, filed on Feb. 15, 2006.

(51) Int. Cl. A61K 31/568; A61K 31/565; A61P 43/00

(52) U.S. Cl. 514/177; 514/182

(57) ABSTRACT

Exposure to a pheromone or an analog thereof will function to accelerate the reestablishment of a disrupted circadian rhythm resultant from cross time zone travel, shift work, exposure to artificial environments or the like. Therapeutic compositions may include pheromones associated with the sex opposite that of the patient being treated, as well as functional analogs of such patients. The therapeutic material may be administered via an olfactory route, and the therapy may be used in conjunction with other therapeutic protocols and materials.

### CROSS-REFERENCE TO RELATED APPLICATIONS

This application claims priority of U.S. Provisional Patent Application Ser. No. 60/773,801 filed Feb. 15, 2006, entitled “Method and Material for Accelerating Reentrainment of a Circadian Rhythm.”

### FIELD OF THE INVENTION

This invention relates generally to therapeutic methods and materials. More specifically, the invention relates to methods and materials for reestablishing circadian rhythms in a patient following a disruption as for example by trans-meridian travel.

### BACKGROUND OF THE INVENTION

Humans, as well as many other animals, have established circadian rhythms which controls a number of mental and physiological processes including, but not limited to, sleep-wake cycles, body temperature, mental acuity, and digestive function, among others. These rhythms may be disrupted by rapid trans-meridian travel, and this phenomenon of disruption is well known under the common term “jet lag.” Likewise, circadian rhythms may be disrupted by other factors such as shift work or prolonged deprivation of exposure to a light/dark cycle pattern. In any event, such disruptions can produce a desynchronization of various internal circadian rhythms and the symptoms of this disruption can include fatigue, irritability, insomnia, intestinal distress, changes in appetite, decrease in mental acuity and/or athletic performance.

Given the prevalence of air travel, jet lag is a significant problem resulting in loss of productivity and is a very significant factor for business travelers, athletes, military personnel and recreational travelers. Likewise, disruption of circadian rhythms is also very significant for shift workers including law enforcement and public safety officers, medical personnel, and various production workers.

Various approaches to the reestablishment of circadian rhythms have been attempted in the past; these include photo therapies wherein patients have been exposed to controlled periods of light and dark, and drug therapies using various combinations of materials including melatonin, sedatives and stimulants. However, none of the prior approaches have been entirely successful in rapidly reestablishing disrupted circadian rhythms. Clearly, there is a need for therapies and methods which can rapidly and safely reestablish circadian rhythms.

### BRIEF DESCRIPTION OF THE INVENTION

Disclosed herein is a method for accelerating the reentrainment of a circadian rhythm in a human patient. The method comprises exposing said patient to a therapeutic material which is a pheromone or a functional analog of a pheromone. The therapeutic material, in some instances, comprises a human pheromone or a functional equivalent thereof. In specific instances, the human pheromone is a pheromone associated with a sexual response. For example, the therapeutic material may comprise a human pheromone generated by a member of the sex opposite that of the patient being treated, or it may comprise a functional analog of that pheromone.

Exposure may, in some instances, be by olfactory pathways. For example, the therapeutic material may be applied into or near the nasal passages of the patient. In certain instances, treatment is carried out on a daily basis for a period of time of at least 3 days. The methodology of the present invention may be extended to species other than humans.

### BRIEF DESCRIPTION OF THE DRAWINGS

FIG. 1 is a graph showing experimental data depicting the acceleration of reentrainment of circadian rhythm in a group of experimental subjects; and

FIG. 2 is a graph showing data depicting body temperatures, as a function of time, for subjects receiving the therapy of the present invention and for a group of control subjects.

### DETAILED DESCRIPTION OF THE INVENTION

The present invention is based upon the finding that stimulation via pheromones and functional analogs can greatly accelerate the reentrainment of circadian rhythms in humans and other species. Most specifically, it has been found that steroidal pheromones are effective in reestablishing circadian rhythms. Most particularly, those steroidal pheromones associated with social interactions in an animal species are effective in reestablishing circadian rhythms in those species. While this discussion refers to “pheromones,” it is to be understood, as is known in the art, that various analogous molecules can have an effect similar to that caused by a particular pheromone. For example, a pheromone molecule may be modified by forming a derivative thereof such as an ester, a salt, an alkylated species, a conjugate or the like. Such modified species can retain pheromonic activity; and in some instances this activity may be increased, extended or otherwise modified. Likewise, new species may be synthesized which include, or mimic, the active portion of the pheromone molecule. All of such species, to the extent that they manifest an activity corresponding qualitatively, if not quantitatively, to the activity of a particular pheromone will be considered functional analogs of that pheromone.

In the instance of humans, pheromones typically associated with the opposite sex have been found effective in reestablishing disrupted rhythms. For example, in the case of female subjects, olfactory stimulation with 4,16-androstadien-3-one has been shown to accelerate reestablishment of body temperature rhythms following eastward jet travel across 6 time zones. Similar results are anticipated utilizing other pheromones and functional analogs. Likewise, in the case of males, it will be shown that exposure to pheromones produced by females, such as 1,3,5(10),16-estratetraen-3-ol, as well as various functional analogs, will result in a similar reentrainment.

Exposure to the pheromone is typically accomplished by olfactory stimulation, and in this regard the subject may inhale a sample of the material or have the material placed proximate, or in, the nasal passages. Other routes of administration such as transdermal or oral administration will likewise be successful.

In accord with the present invention, there are provided therapies which can eliminate or alleviate problems of jet lag. Likewise, the methods and materials of the present invention may be utilized by shift workers and others experiencing disrupted circadian patterns. The invention will be explained with reference to a particular experimental series which evaluated and demonstrated the benefits of pheromones in enhancing reentrainment of circadian rhythms.

This experimental involved 15 subjects who were traveling from Detroit, Michigan to Vienna, Austria by air, a journey which spanned 6 time zones. The subjects comprised 8 women and 5 men aged 18-19 and 2 women aged 37 and 39. Two groups of compositions were prepared. The first comprised an experimental formulation containing 20 micrograms of 4,16-androstadien-3-one dissolved in a mixture of mineral oil and clove oil. A second composition comprising a control/placebo consisted of the mineral oil and clove oil. The 2 compositions were each packaged in 5 ml and supplied to selected test subjects along with cotton-tipped applicators. The test and control materials were randomly distributed, and participants were unaware of which composition they had obtained.

Upon arrival in Vienna, each participant was directed to place a small amount of their particular composition directly beneath their nostrils with a new cotton applicator immediately upon awakening each morning. The participants were directed to allow the compositions to remain on their skin for at least an hour.

External temperature monitors (iButton, Inc., Pittsburgh, Pa.) were affixed to each of the subjects with a gauze protective layer between the skin and the monitoring device. The monitors were covered with neoprene material for insulation against environmental temperature interference and held in place with a waterproof medical bandage which was changed every 3 days or as necessary. The monitoring devices were set to acquire temperature every 10 minutes throughout the entire period of the experiment and for a 3-day period prior to travel. Participants also kept daily event logs to monitor the timing of activities that could influence temperature data such as sleep and wake times, showers, exercise and naps. Their logs were correlated with unexpected changes in body temperature and provided a means by which the timing of data analysis could be verified.

Participants agreed to comply with standard sleep and wake times for at least 3 days prior to traveling, waking at 0600 h and being in bed by 2200 h. The same schedule was maintained, on local time, the entire time the participants were in Vienna. During the period of reentrainment, participants were asked to avoid napping, and daily group activities were provided to maintain wakefulness.

Data were analyzed to determine the length of time needed for core temperature rhythms to reentrain with the new time schedule, returning to a phase angle of entrainment similar to that recorded before travel. Two persons who were blind to the conditions of the participants independently determined the day on which each participant's body temperature rhythm showed a consistent, entrained rhythm, as determined by using printed actograms. Consistency was defined as the presence of peaks or troughs in temperature data that occurred for at least 3 consecutive days at approximately the same time of day (within 20 minutes) and that were not associated with external events such as exercise or bathing. Between-groups comparisons were conducted with independent sample t-tests.

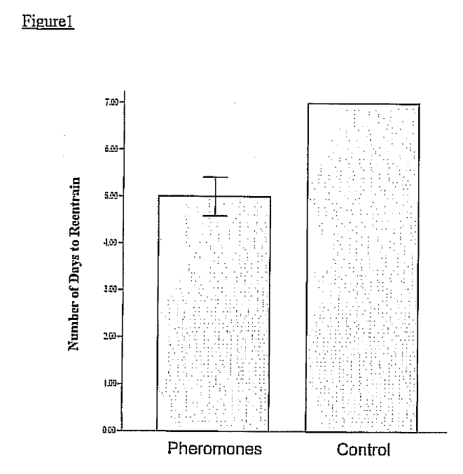

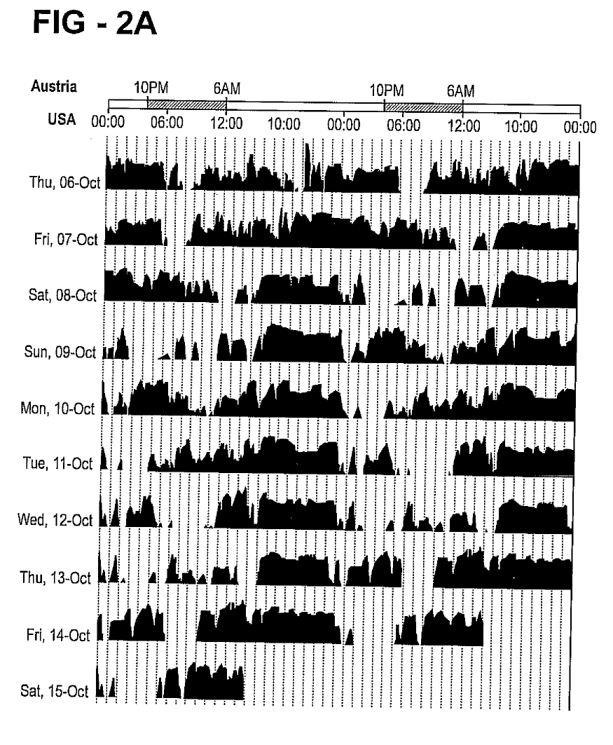

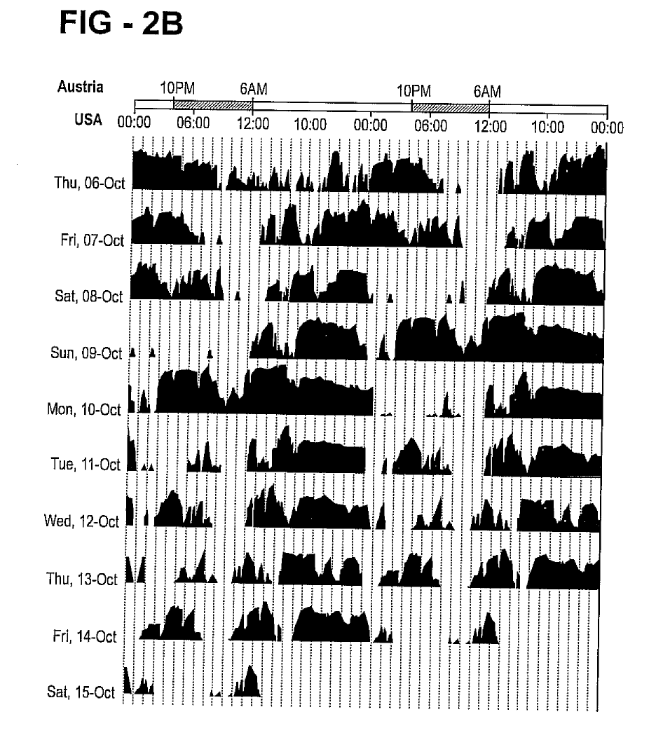

The data indicate a significant effect of pheromone exposure on length of time to reestablish stable body temperature rhythms in women, but not in men. Women who were exposed to the pheromone reentrained significantly faster than women in the control group who were exposed only to the control vehicle. In this regard, FIG. 1 shows a graphic representation of these results. FIG. 2 displays typical actograms for women in the experimental and control groups. As is demonstrated in this experimental series, the active material acted as a true pheromone, affecting only the sex opposite that from which the substance is produced in greatest quantities. Men who were exposed to the active composition showed no effects on reentrainment rate of their body temperature rhythms. Substitution of a female-derived pheromone, or its functional analog, is predicted to show the opposite effect, namely fostering reentrainment of circadian rhythm in men but not in women.

The foregoing demonstrates that pheromone therapy will effectively foster the reliable reestablishment of circadian rhythms in humans. Corresponding results are anticipated in other species. As discussed above, various other therapies and methods have been proposed for reentraining circadian rhythms. These include photo therapies wherein subjects are exposed to bright light during periods corresponding to daylight in the time zone in which rhythms are to be reestablished. Likewise, administration of melatonin or its functional analogs during periods of darkness in the selected time zone has also been shown to have some effect. The pheromone therapy of the present invention may be implemented in combination with various of such other therapies. For example, prior to traveling to a particular time zone, a patient may combine pheromone therapy with light and/or melatonin therapy for purposes of “presetting” his or her circadian rhythm to the anticipated destination. Likewise, the therapy of the present invention may be utilized in situations where a normal circadian rhythm must be maintained in an artificial environment such as a submarine vessel, an arctic or antarctic environment, or a spacecraft.

While the present invention has been described with reference to particular pheromone and pheromone analog materials, it is to be understood that in view of the teaching presented herein, implementation of the therapy with other materials and functional analogs will be readily apparent to those of skill in the art. Also, various dosing regimens, protocols and concentrations will be readily apparent to, and implemented by, those of skill in the art. The foregoing discussion, description and examples are illustrative of specific embodiments of the invention, but are not meant to be limitations upon the practice thereof. It is the following claims, including all equivalents, which define the scope of the invention.

1. A method for accelerating reentrainment of a circadian rhythm in a human patient, said method comprising:
exposing said patient to a therapeutic material comprising a pheromone or an analog of a pheromone.

2. The method of claim 1, wherein said therapeutic material comprises a human pheromone or a functional analog of said human pheromone.

3. The method of claim 2, wherein said human pheromone is associated with a sexual response in said patient.

4. The method of claim 2, wherein said human pheromone is a pheromone generated by a member of the opposite sex of said patient.

5. The method of claim 1, wherein said therapeutic material is 4,16-androstadien-3-one or a functional analog thereof.

6. The method of claim 1, wherein said therapeutic material is 1,3,5(10),16-estratetraen-3-ol or a functional analog thereof.

7. The method of claim 1, wherein said step of exposing said patient to said therapeutic material comprises exposing said patient to said therapeutic material so as to cause olfactory stimulation of said patient by said therapeutic material.

8. The method of claim 1, wherein the step of exposing said patient to said therapeutic material comprises exposing said patient to said therapeutic material on a daily basis for at least three days.

9. The method of claim 1, including the further step of exposing said patient to illumination having a time-based profile corresponding to the time-based profile of the circadian rhythm to which said animal is to be reentrained.

10. A method for accelerating reentrainment of a circadian rhythm in an animal, said method comprising:
exposing said animal to a pheromone.
